(19) United States
(12) Patent Application Publication
(10) Pub. No.: US 2016/0029970 A1
(43) Pub. Date: Feb. 4, 2016

(54) APPARATUS AND METHOD FOR DETERMINING VALIDITY OF BIOLOGICAL DATA

(71) Applicant: LG CNS CO., LTD., Seoul (KR)

(72) Inventor: Si Young PARK, Seoul (KR)

(73) Assignee: LG CNS CO., LTD., Seoul (KR)

(21) Appl. No.: 14/448,713

(22) Filed: Jul. 31, 2014

(51) Int. Cl. A61B 5/00; G01G 19/50; A61B 5/0205; A61B 5/107; A61B 5/145

(52) U.S. Cl. CPC A61B 5/7221; A61B 5/1072; A61B 5/14532; A61B 5/02055; G01G 19/50; A61B 5/0002; A61B 5/6898; A61B 5/021

(57) ABSTRACT

A method that includes acquiring a first biological data of a user from a measuring device and determining whether the acquired first biological data is within a valid data range, such that the valid data range includes at least one valid section, and a validity time period is set for each section of the at least one valid section. The method further includes transmitting the first biological data to a terminal when the acquired first biological data is within the valid data range.

### BACKGROUND OF THE INVENTION

1. Field of the Invention

The present invention relates, in general, to an apparatus and method for determining the validity of biological data of a user.

2. Description of the Related Art

As well known in the art, biological data of a user including: body weight, height, blood glucose level, and body temperature are used to determine the user's health status. For example, medical physicians periodically check the blood pressure of their hypertension patients, and blood glucose levels of diabetes patients in order to determine the progress of the patients' diseases.

Recently, with the wide distribution of home-based self-assessment devices enabling easy measurement of a user's biological data, the users can now easily measure and transmit their own self-measured biological data to physician terminals, thereby reducing the number of actual visits to medical clinics.

However, since the biological data is often measured by non-professional users via a home-based measuring device, the biological data items obtained therefrom may not be fully reliable or otherwise valid. In other words, the acquired biological data may not be accurate due to a lack of operation skills on the part of the users, malfunctioning of the measuring devices, etc. Accordingly, the physicians working with the biological data being directly transmitted to them by many users may incur a heavy burden with respect to how to accurately determine the validity of such data. Accordingly, under the current situation with many home-based measuring devices available on the market, an effective solution to enhance the validity of the biological data measured by users using home-based or other measuring devices is needed.

### SUMMARY

Accordingly, the present invention has been made keeping in mind the above problems occurring in the prior art, and a feature of some embodiments is to provide an apparatus and a method for determining the validity of the biological data measured by a measuring device capable of determining the validity of the biological data with improved accuracy, thereby reducing or perhaps preventing the risk of misdiagnosis of the users' health conditions.

Another feature is to provide an apparatus and a method for determining the validity of the biological data, from numerous users, capable of reducing the burden of physicians resulting from the need for them to directly determine the validity of the biological data.

A further feature is to provide an apparatus and a method for determining the validity of the biological data capable of improving the reliability of the measuring devices; thereby promoting the distribution of the home-based measuring devices.

One aspect includes a method for determining validity of biological data via a validity-determining apparatus, including functions of acquiring a first biological data of a user measured by a measuring device; determining whether or not the acquired first biological data is within a valid range regarding the biological data; and transmitting the first biological data to a physician terminal when the acquired first biological data is within the valid range; wherein the valid range includes at least one valid section, and a validity period is set for each of the at least one valid section, respectively.

The biological data may comprise body weight, height, blood glucose level, blood pressure or body temperature of the user. The transmission process may comprise updating the validity period set in the valid section including the first biological data. The method may further include outputting a message requesting communication with the physician terminal when the first biological data is not included within the valid range.

The method for determining the validity of the biological data may further include updating the valid range based on the first biological data when information, indicating that the first biological data is valid, is received from the physician terminal although the first biological data is not included within the valid range.

The updating process may comprise adjusting the valid range to include a new valid section including the first biological data, and setting a validity period for the new valid section.

The method for determining the validity of the biological data may further include outputting a message requesting a re-measurement of the biological data when the first biological data is not included within the valid range.

The method for determining the validity of the biological data may further include: determining whether or not the user's second biological data measured by the measuring device is acquired, after outputting the message; and outputting a message requesting repair or replacement of the measuring device when the second biological data is not acquired within a preset time period.

The method for determining the validity of the biological data may further include eliminating a first valid section from the valid range when the biological data is not acquired during the validity period set for the first valid section.

The validity period set for each of the at least one valid section may be set based on the number of the biological data already included in each of the valid section, respectively.

The method for determining the validity of the biological data may further include generating the valid range based on other users' biological data when the biological data of the user already measured is not present, wherein the other users have characteristics corresponding to those of the user.

In another embodiment of the present invention, there is provided a validity-determining apparatus for determining the validity of the biological data may include: a memory for storing at least one program; and a processor, being controlled by the at least one program, for performing a method for validity determination; wherein the method for validity determination may comprise: acquiring a first biological data of a user measured by a measuring device; determining whether or not the acquired first biological data is within a valid range regarding the biological data; and transmitting the first biological data to a physician terminal when the acquired first biological data is within the valid range; wherein the valid range includes at least one valid section, and a validity period is set for each of the at least one valid sections, respectively.

The biological data may comprise body weight, height, blood glucose level, blood pressure or body temperature of the user.

The transmission process may comprise updating the validity period set in the valid section including the first biological data.

The method for determining the validity of the biological data may further include outputting a message requesting communication with the physician terminal when the first biological data is not included within the valid range.

The method for determining the validity of the biological data may further include updating the valid range based on the first biological data when information, indicating that the first biological data is valid, is received from the physician terminal although the first biological data is not included within the valid range.

The updating process may comprise adjusting the valid range to include a new valid section including the first biological data, and setting a validity period for the new valid section.

The method for determining the validity of the biological data may further include outputting a message requesting a re-measurement of the biological data when the first biological data is not included within the valid range.

The method for determining the validity of the biological data may further include: determining whether or not the user's second biological data measured by the measuring device is acquired, after outputting the message; and outputting a message requesting repair or replacement of the measuring device when the second biological data is not acquired within a preset time period.

The method for determining the validity of the biological data may further include eliminating a first valid section from the valid range when the biological data is not acquired during the validity period set for the first valid section.

The validity period set for each of the at least one valid section may be set based on the number of the biological data already included in each of the valid section, respectively.

The method for determining the validity of the biological data may further include generating the valid range based on other users' biological data when the biological data of the user already measured is not present, wherein the other users have characteristics corresponding to those of the user.

A non-transitory computer program for performing the validity determination of the biological data may be recorded on a computer-readable recording medium.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a schematic diagram illustrating general components utilized for transmitting biological data measured by a measuring device to a physician terminal;

FIG. 2 is a flowchart depicting a method for determining the validity of biological data according to an embodiment of the present invention;

FIG. 3 is diagram showing an example of a valid rage of blood pressure;

FIG. 4 is a flowchart depicting a method for determining validity of the biological data according to another embodiment of the present invention;

FIG. 5 is a diagram showing a valid range of blood pressure updated from that of FIG. 3;

FIG. 6 is a diagram showing a valid range of blood pressure also updated from that of FIG. 3;

FIG. 7 is flowchart depicting a method for determining the validity of the biological data according to a further embodiment of the present invention;

FIGS. 8A, 8B and 8C illustrate messages being output by a user terminal; and

FIG. 9 is a block diagram illustrating a validity-determining apparatus according to an embodiment of the present invention.

### DETAILED DESCRIPTION

Presented herein are exemplary embodiments of the present invention and will be described in detail with reference to the accompanying drawings. While the present invention will be described in conjunction with exemplary embodiments thereof, it is to be understood that the present description is not intended to limit the present invention to those exemplary embodiments. Instead, the present invention is intended to cover not only the exemplary embodiments, but also various alternatives, modifications, equivalents and other embodiments that may be included within the spirit and scope of the present invention as defined by the appended claims.

Throughout the specification, the same reference numerals will generally refer to the same or similar parts. According to exemplary embodiments, the word “unit” may refer to a software component or hardware component such as an FPGA or ASIC capable of carrying out a function or an operation. However, “unit” is not limited to hardware or software. A unit may be configured to reside in an addressable storage medium or to drive one or more processors. Units may refer to software components, object-oriented software components, class components, task components, processes, functions, attributes, procedures, subroutines, program code segments, drivers, firmware, microcode, circuits, data, databases, data structures, tables, arrays or variables. A function provided by a component and unit may be a combination of smaller components and units, and may be combined with others to compose large components and units, or further divided into units along with other components.

The apparatus and the method for determining the validity of the biological data according to an embodiment of the present invention can determine the validity of the biological data measured by a measuring device with generally improved accuracy and thus can reduce or prevent misdiagnosis of a user's health status.

The apparatus and the method for determining the validity of the biological data according to an embodiment of the present invention may also be implemented to reduce the burden upon physicians in having to directly determine the validity of the biological data of a large number of users.

The apparatus and the method for determining the validity of the biological data according to an embodiment of the present invention can improve the reliability of such measuring devices, thereby promoting the distribution of home-based measuring devices.

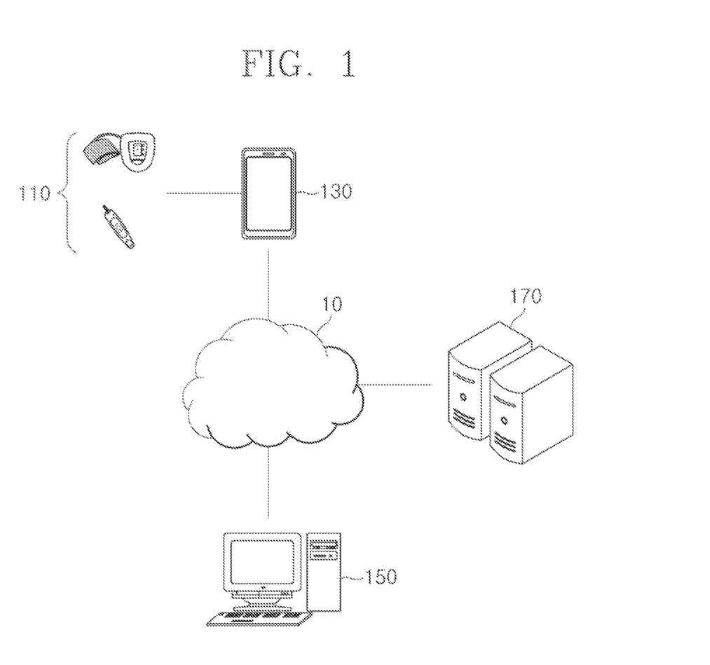

FIG. 1 is a schematic diagram illustrating general components utilized for transmitting biological data measured by a measuring device to a physician terminal. As shown in this figure, the measuring device 110 may be used to measure biological data of a user. In general, the measuring device 110 may be implemented using most any type of device capable of measuring a physical characteristics of a user. Examples of such a measuring device include a scale, a sphygmomanometer, a blood glucose meter, a thermometer, and the like.

A user terminal 130 is shown acquiring the biological data of a user measured by the measuring device 110. The user terminal 130 may include any of a variety of devices capable of performing communications via a network 10. Examples of such devices for the user terminal include a smart phone, a PDA, a tablet PC, and a desk top computer, among others.

The user terminal 130 and the measuring device 110 may be operatively coupled via communication link, such as a near field communication (NFC) which includes various communication methods including Bluetooth communication, NFC communication, RFID communication, or a Zigbee communication, among others.

Although FIG. 1 illustrates that the measuring device 110 and the user terminal 130 are two separate devices, the measuring device 110 may be included within the user terminal 130 if so desired. For example, a smart phone capable of measuring heartbeats of a user using an application installed therein may be an example of the user terminal 130 including the measuring device 110.

The user terminal 130 may transmit the biological data acquired by the measuring device 110 via a server 170 connected through the network 10, and the server 170 may store the biological data received from the user terminal 130, and transmit it to a physician terminal 150 connected through the network 10. Various embodiments presented herein will be described with regard to a user terminal providing data to a physical terminal. However, alternative embodiments may also be implemented where one terminal provides such data to a second terminal.

The physician terminal 150 may output the biological data to be confirmed by the physician, and the physician may diagnose the user's health status based on the biological data.

Some embodiments omit server 170. In these embodiments, the user terminal 130 may directly transmit the biological data to the physician terminal 150.

As described above, a physician alone may have difficulty in directly determining the validity of the biological data transmitted from users. Accordingly, the validity-determining apparatus, such as those presented herein, may facilitate the determination of the validity of such biological data measured by the determining apparatus 110 and allow only the biological data determined to be valid as such to be used for a health diagnosis or other reason.

The determining apparatus according to an embodiment of the present invention may be included within the user terminal 130, or may be included within the server 170 depending on the embodiments. That is, the determining apparatus may be included within the user terminal 130 or the server 170, thereby determining the validity of users' biological data, and transmit the biological data determined as valid to the physician terminal 150.

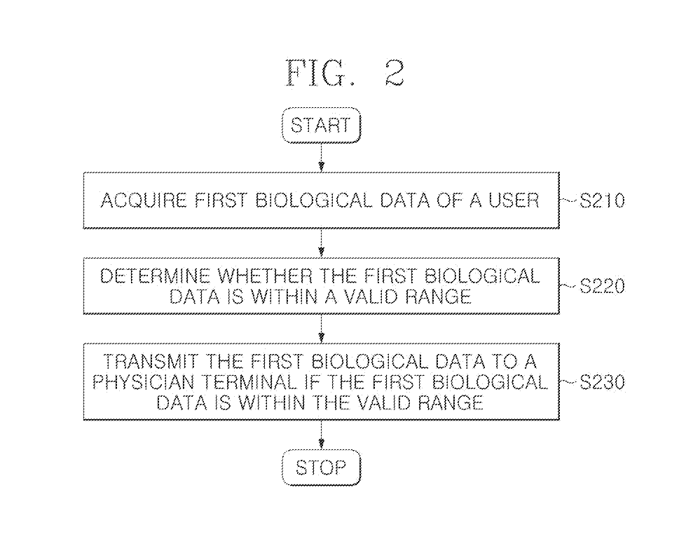

The detailed operation of the determining apparatus will be explained herein below referring to FIG. 2.

FIG. 2 is a flowchart depicting a method for determining the validity of biological data according to an embodiment of the present invention. Block S210 indicates that the determining apparatus acquires the first biological data measured by the measuring device 110. As described above, when the determining apparatus is included within the user terminal 130, the determining apparatus may acquire the first biological data from the measuring device 110, whereas when the determining apparatus is included within the server 170, the determining apparatus may additionally or alternatively acquire the first biological data from the user terminal 130.

Block S220 determines whether the first biological data is within a valid range. The valid range refers to a range serving as a basis for determining whether the first biological data is a valid value or not, and the valid range may include upper and lower limits (e.g., thresholds) of the biological data. Additional or alternatively, the valid range (e.g., valid data range) may be divided or partitioned in terms of one or more of the valid sections, and for each of the valid sections, there may be set a validity period. Each of the valid sections may include upper and lower limits, and the validity period refers to a time period within which a valid section may be included within the valid range.

When the biological data already measured by a user is not present, the valid range may be generated based on other users' biological data, having characteristics corresponding to those of the user. The characteristics of a user may be directly input by the user or may be input by a physician or other source.

For example, for a user aged 30 that is 180 cm tall, weighing 70 kg, and symptoms of hypertension, the valid range of the user may be generated based on the statistical distribution of blood pressure data of other users having the age, height, and body weight generally corresponding to those of the user. For example, if other users' blood pressure data reveals a distribution of 10% from 121 mmHg to 130 mmHg, 40% from 131 mmHg to 140 mmHg, and 50% from 151 mmHg to 160 mmHg, the determining apparatus determines an first valid section of from 121 mmHg to 130 mmHg, a second valid section of from 131 mmHg to 140 mmHg, and a third valid section of from 151 mmHg to 160 mmHg. Then, a corresponding validity period may be set for each of the first, second, and third valid sections, thereby identifying a valid range.

Alternatively, the valid range may be generated based on acquired biological data when the measuring device 110 is correctly used according to the operation manual or other verifiable manner. That is, the upper and lower limits of the valid range may be determined based on the distribution of the biological data acquired by correct use of the measuring device 110, and the valid range may be divided into at least one valid section according to any standard set therein. In yet another alternative, the valid range may be generated via direct input by a physician or other source.

Block S230 is for the determining apparatus to transmit the first biological data to the physician terminal 150 when the first biological data is within the valid range. The determining apparatus according to an embodiment of the present invention determines the validity of the first biological data based on a predetermined standard instead of immediately transmitting the first biological data to the physician terminal 150 upon acquiring the first biological data from the measuring device 110, thus facilitating the reduction of the physician's burden of direct determination of the validity of the first biological data.

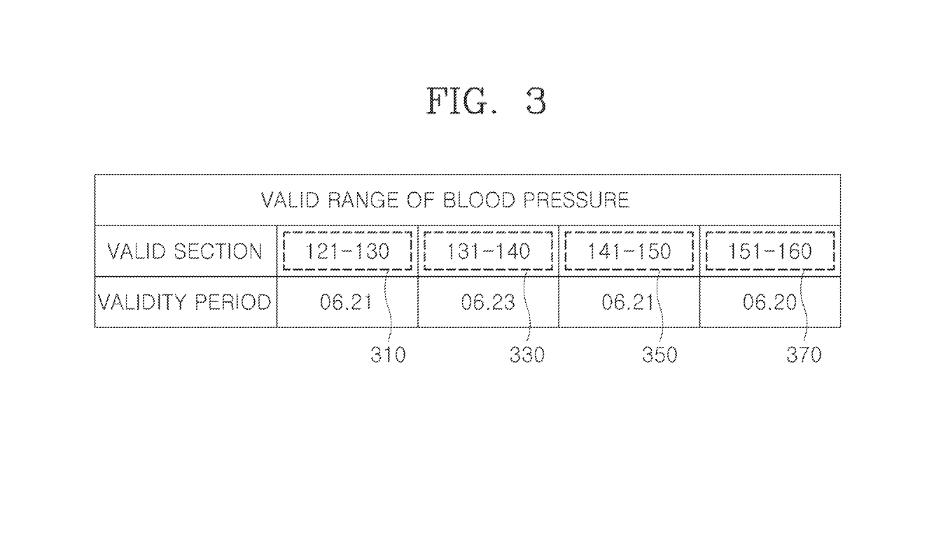

FIG. 3 is diagram showing an example of a valid rage of blood pressure. In this figure, the example includes the upper limit of 160 mmHg and a lower limit of 121 mmHg as a valid range, in which the valid range includes an first valid section 310 of from 121 mmHg to 130 mmHg, a second valid section 330 of from 131 mmHg to 140 mmHg, a third valid section 350 of from 141 mmHg to 150 mmHg, and a fourth valid section 370 of from 151 mmHg to 160 mmHg.

When the measured blood pressure is in the range of from 121 mmHg to 160 mmHg the determining apparatus determines that the user's measured blood pressure is valid, and transmits it to a physician terminal 150, for example.

When the acquired user's biological data is not within the validity period of any of the valid sections (i.e., valid sections 310, 330, 350, 370), the determining apparatus may update the valid range by eliminating the valid section from the valid range. For example, when the biological data included within the fourth valid section 370 ranged from 151 mmHg to 160 mmHg is not acquired, the determining apparatus may eliminate the fourth valid section 370 from the valid range.

The validity period to be set for each of the at least one valid section(s) may be set based on the number of the biological data items already included in each of the valid sections. More specifically, when the number of the biological data included within the first valid section 310 is smaller than that included within the second valid section 330, the validity period corresponding to the first valid section 310 may be set shorter than that for the second valid section 330.

The reason for setting a validity period based on the number of the biological data items already included within the valid section is because the biological data belonging to a valid section including a greater number of the biological data is more likely to be valid. Accordingly, a valid range including a greater number of the biological data is given a longer validity period.

In contrast, the biological data belonging to a valid section including a lesser number of the biological data may be one measured exception and is thus given a shorter validity period for its valid section, thereby compelling more frequent checkups on the validity of the biological data acquired after their expiration dates.

Accordingly, when a user's biological data is not acquired within the validity period set for any one of the valid sections 310, 330, 350, 370, the determining apparatus according to an embodiment of the present invention may eliminate the valid section from the valid range, thereby primarily determining the biological data as invalid even when the biological data belonging to the very valid section is acquired at a later time.

Although FIG. 3 indicates the validity period in terms of months and days, this is only for the purpose of example, and other time periods may alternatively or variably be used.

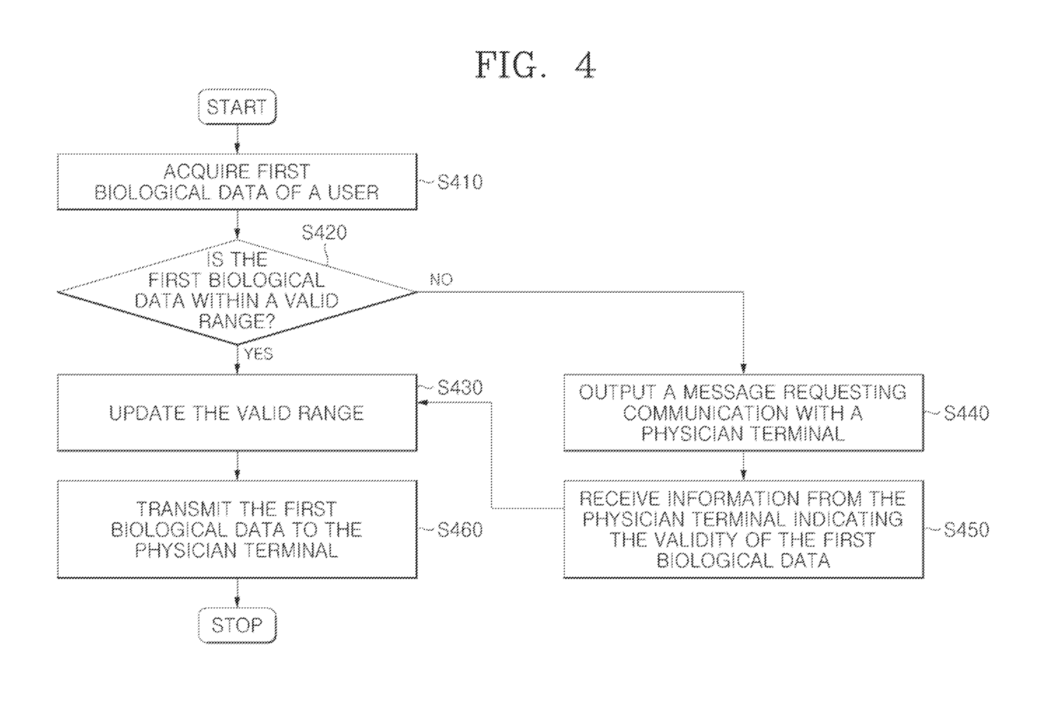

FIG. 4 is a flowchart depicting a method for determining validity of the biological data according to another embodiment of the present invention. Block S410 acquires the user's first biological data. Block S420 determines whether the first biological data belongs to the valid range.

If the data is valid, processing flows to block S430 to renew the valid range. More specifically, the determining apparatus may extend the validity period preset in the valid section which includes valid sections, in any one of which the first biological data is included. This is because when the validity period is not extended, the valid section may be eliminated from the valid range due the lapse of its validity period.

When the first biological data is not included within the valid range, control flows to block S440 where a message requesting communication with the physician terminal 150 is output. It is possible that the first biological data may be valid data although the first biological data is not included within the valid range, and thus, the validity of the first biological data can be reconfirmed by performing the communication with the physician terminal 150.

When the determining apparatus is included within the server 170, the determining apparatus may transmit a control message to control the user terminal 130 so that a message requesting communication with the physician terminal 150 may be output.

Block S450 renews the valid range by returning to block S430 when the information indicating the validity of the first biological data is received from the physician terminal 150. More specifically, since the first biological data is confirmed as valid by the physician, although it is not included within the valid range, the determining apparatus may adjust the valid range to include a new valid section including the first biological data and set up a validity period corresponding to the new valid section, thus updating the valid range data. Here, the number of the biological data to be included within the new valid section is one and thus the validity period to be set for the new valid section will be relatively short.

In contrast, when the information confirming the validity of the first biological data is not received from the physician terminal 150, the determining apparatus may determine that an error has occurred in the measuring device 110 thereby outputting a message requesting the repair or replacement of the measuring device 110.

Block S460 transmits the first biological data determined to be included within the valid range or the first biological data determined by the physician terminal 150 as valid to the physician terminal 150.

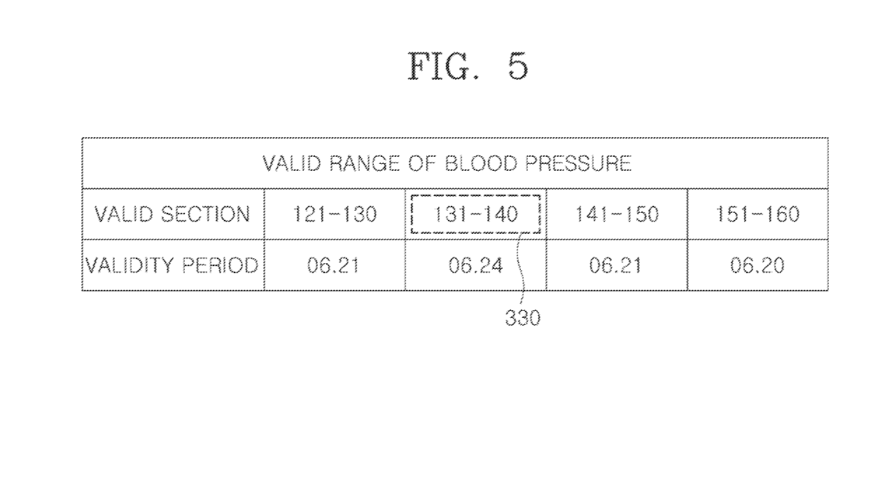

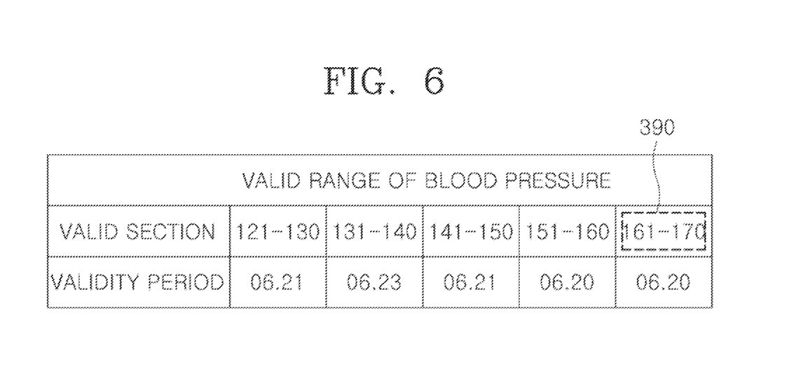

The exemplary methods for updating valid ranges when the first biological data is determined as valid will be described below with reference to FIGS. 5 and 6.

FIG. 5 is a diagram showing a valid range of blood pressure updated from that of FIG. 3 when a user's first biological data is within the valid range, and FIG. 6 is a diagram showing a valid range of blood pressure also updated from that of FIG. 3, when information, indicating that the first biological data is valid, is received from the physician terminal although the user's first biological data is not included within the valid range.

First, when the first biological data is determined by the determining apparatus to be within the valid range, the determining apparatus may extend the validity period set for the second valid section 330 as illustrated in FIG. 5. Referring to FIGS. 3 and 5, the validity period firstly set for the second valid section 330 was extended from June 23 to June 24.

Then, when the first biological data is determined as valid by the physician, although the first biological data is not included within the valid range by the determining apparatus, the determining apparatus may adjust the valid range to include a fifth valid section 390 including the first biological data, and set up a validity period corresponding to the fifth valid section 390. Referring to FIG. 6, it can be seen that the fifth valid section 390 including the upper limit of 170 mmHg and the lower limit of 161 mmHg was generated, and that the validity period is set to expire on June 20.

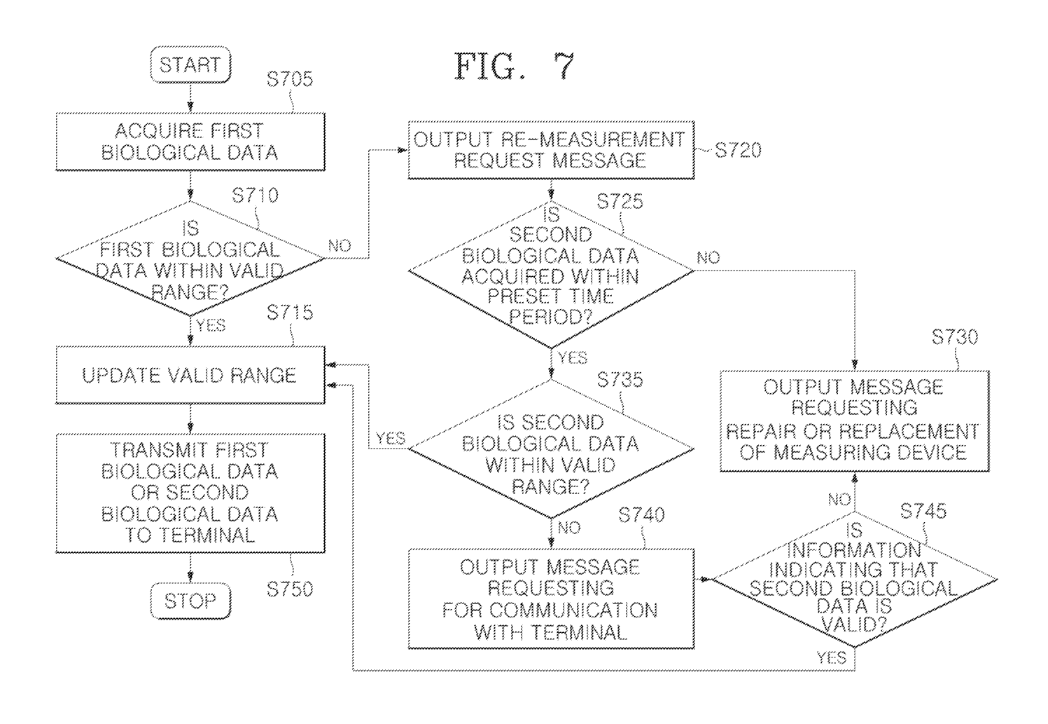

FIG. 7 is flowchart depicting a method for determining the validity of the biological data according to a further embodiment of the present invention. Block S705 acquires the user's first biological data. Block S710 determines whether the user's first biological data is within the valid range.

When the first biological data is within the valid range, in block S715, the determining apparatus renews the valid range. As described above, the determining apparatus may extend the validity period set for the valid section including the first biological data.

When the first biological data is not included within the valid range, at block S720, the determining apparatus outputs a message requesting a re-measurement of biological data. The determining apparatus may output an operation manual for the measuring device 110 while outputting a re-measurement request message.

When the determining apparatus is included within the server 170 (FIG. 1), the determining apparatus may transmit a control message to the user terminal 130 so that a message requesting the re-measurement of the biological data can be output by the user terminal 130.

At block S725, upon outputting the re-measurement request message, there is a determination as to whether the user's second biological data is acquired within a preset time period. The preset time period may be already stored in the determining apparatus or may be modified by a manager of the determining apparatus.

When the second biological data is not acquired within the preset time period, at block S730, the determining apparatus may determine that an error has occurred to the measuring device 110 and thus output a message requesting the repair or replacement of the measuring device 110.

When the second biological data is acquired within the predetermined time period, at block S735, the determining apparatus determines whether the second biological data is within the valid range. When the second biological data is within the valid range, at block S715, the determining apparatus renews the valid range. More specifically, the determining apparatus may extend the validity period for a valid section including the second biological data. When the second biological data is not within the valid range, at block S740, the determining apparatus outputs a message requesting communication with the physician terminal 150.

Block S745 determines whether any information indicating the validity of the second biological data is received from the physician terminal 150. When information indicating the validity of the second biological data is received from the physician terminal 150, at block S715, the determining apparatus renews the valid range. More specifically, the determining apparatus adjusts the valid range to include a new valid section including the first biological data, and sets up a validity period corresponding to the new valid section.

On the other hand, when the information indicating the validity of the second biological data is not received from the physician terminal 150, at block S730, the determining apparatus may determine that an error has occurred to the measuring device 110, thereby outputting a message requesting the repair or replacement of the measuring device 110.

Block S750 transmits to the physician terminal 150 the first biological data within the valid range, the second biological data within the valid range, or the second biological data confirmed as valid by the physician although it is not within the valid range.

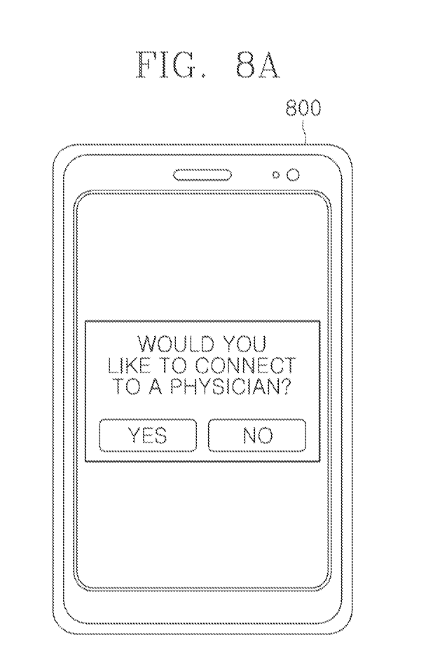

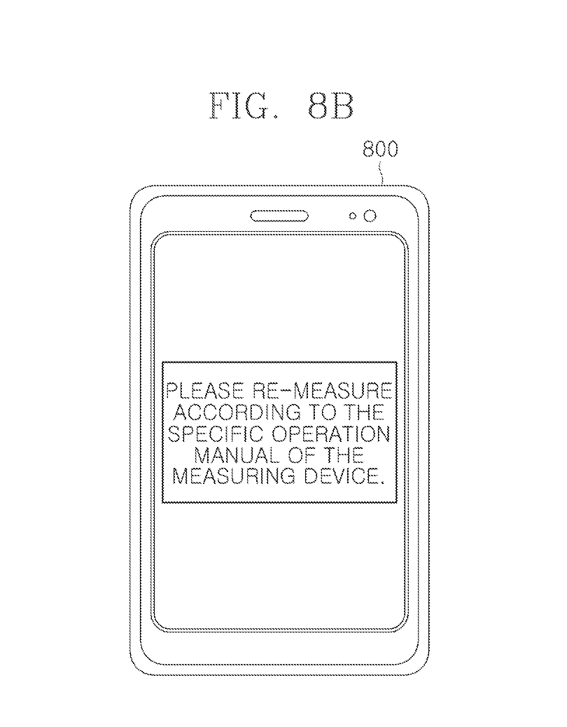

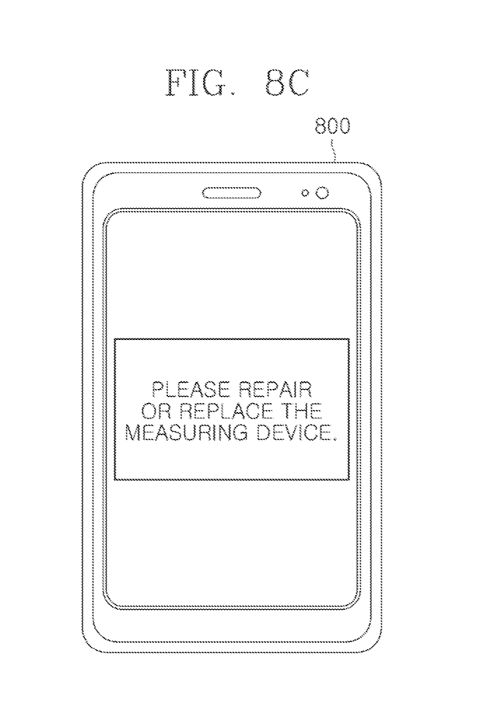

FIGS. 8A, 8B and 8C illustrate messages being output by the user terminal 800. In general, the determining apparatus according to an embodiment of the present invention may output various messages via a user terminal 800 for enhanced accuracy of the biological data, for example.

First, as shown in FIG. 8A, the determining apparatus may output a message requesting communication with the physician terminal 150 when the biological data measured by the measuring device 110 is not within a valid range. The user may communicate with the physician by clicking on the ‘yes’ or ‘no’ icon button indicated on the user terminal 800.

Additionally, as shown in FIG. 8B, the determining apparatus may output a message requesting a re-measurement of the biological data through the user terminal 800 when the biological data measured by the user is not within the valid range. Furthermore, the determining apparatus may also output information regarding the specific user manual for the measuring device 110 through the user terminal 800 along with the re-measurement request message.

Lastly, as shown in FIG. 8C, the determining apparatus, upon determining the occurrence of an error to the measuring device 110, may output a message requesting the repair or replacement of the measuring device 110 through the user terminal 800.

The messages illustrated in FIGS. 8A through 8C are only exemplary, and the determining apparatus may output the corresponding messages by voice using a speaker or via other various methods.

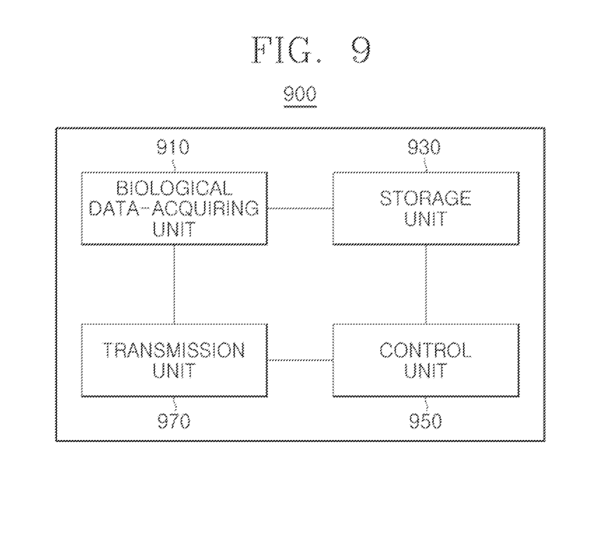

FIG. 9 is a block diagram illustrating a validity-determining apparatus according to an embodiment of the present invention. In this figure, the validity-determining apparatus 900 includes a biological data-acquiring unit 910, a storage unit 930, a control unit 950 and a transmission unit 970.

The various units shown in FIG. 9 are functional blocks being performed by a respective unit, and they may be embodied in one or more processors, and may operate under the control one or more programs stored in memory.

The biological data-acquiring unit 910 acquires a user's biological data measured by the user via the measuring device 110. When the validity-determining apparatus 900 according to an embodiment of the present invention is included within the user terminal 130 illustrated in FIG. 1, the biological data-acquiring unit 910 may acquire the biological data from the measuring device 110. On the other hand, when the validity-determining apparatus 900 is included within the server 170 illustrated in FIG. 1, the biological data-acquiring unit 910 may acquire the biological data from the user terminal 130.

The storage unit 930 stores the valid range generated by the control unit 950. The valid range stored in the storage unit 930 may be renewed by the control unit 950.

The control unit 950 is generally configured to perform the various operations presented herein and show in the various flowcharts, for example. For instance, the control unit may determine whether a user's biological data acquired by the biological data-acquiring unit 910 is within the valid range. The control unit 950 may also generate a valid range and store it in the storage unit 930.

The control unit 950 may update the valid range when the biological data is within the valid range, or when the biological data is confirmed as being within the valid range by a physician although it is not within the valid range. Additionally, the control unit 950 may eliminate any one of the valid sections from the valid range when the any one of the valid section is not acquired within the validity period set for the valid section.

The transmission unit 970 transmits the biological data included within the valid range or the biological data confirmed as valid by the physician to the physician terminal 150. According to various embodiments, the biological data, although confirmed as valid, may not be necessarily transmitted to the physician terminal 150 because the biological data confirmed as valid by the physician has already been transmitted to the physician terminal 150.

Additionally, when the validity-determining apparatus 900 is included within the server 170 as illustrated in FIG. 1, the transmission unit 970 may transmit a control message for controlling the user terminal 130 to the user terminal 130 so that a message requesting the re-measurement of the biological data and communication with the physician terminal 150, etc., may be output by the user terminal 130.

Additionally, when the validity-determining apparatus 900 is included within the user terminal 130, the validity-determining apparatus 900 may further include an output unit. The output unit may include various output devices such as a display, a speaker, a printer, etc., within the range being understood to a skilled person in the art.

The output unit may output a message requesting re-measurement, a message requesting communication with the validity-determining apparatus 900 according to an embodiment of the present invention, etc., under the control of the control unit 950.

The embodiments of the present invention described above may be drafted into computer-executable programs, and may be embodied within a conventional digital computer for operating the program using a computer-readable recording medium.

The computer-readable recording medium may include nontransitory storage media such as a magnetic storage medium (e.g., ROMs, floppy disks, hard disks, etc.), an optically readable medium (e.g., CD ROMs, DVDs, etc.), and a carrier wave (e.g., transmission via the internet).

Although preferred embodiments of the present invention have been described for illustrative purposes, those skilled in the art will appreciate that various modifications, additions and substitutions are possible, without departing from the scope and spirit of the invention as disclosed in the accompanying claims. Therefore, the embodiments of the present invention are disclosed only for illustrative purposes and should not be construed as limiting the present invention.

### DESCRIPTION OF THE ELEMENTS IN THE DRAWINGS

110: measuring device
130, 800: user terminal
150: physician terminal
170: server
900: validity-determining apparatus
910: biological data-acquiring unit
930: storage unit
950: control unit
970: transmission unit

What is claimed is:

1. A data transmission method, comprising:
acquiring a first biological data of a user from a measuring device;
determining whether the acquired first biological data is within a valid data range, wherein the valid data range includes at least one valid section, and a validity time period is set for each section of the at least one valid section; and
transmitting the first biological data to a terminal when the acquired first biological data is within the valid data range.

2. The method of claim 1, wherein the biological data is at least one of body weight, height, blood glucose level, blood pressure, or body temperature.

3. The method of claim 1, further comprising:
updating the validity time period set for at least one valid section of the at least one valid section.

4. The method of claim 1, further comprising:
outputting a message requesting communication with the terminal when the first biological data is not included within the valid data range.

5. The method of claim 1, further comprising:
updating the valid data range based on the first biological data when information, indicating that the first biological data is valid, is received from the terminal, wherein the updating occurs despite the first biological data being not included within the valid data range.

6. The method of claim 5, wherein the updating comprises adjusting the valid data range to include a new valid section including the first biological data, and setting a validity time period for the new valid section.

7. The method of claim 1, further comprising:
outputting a message requesting a re-measurement to obtain second biological data when the first biological data is not included within the valid data range.

8. The method of claim 7, further comprising:
determining whether the second biological data measured by the measuring device is acquired, after outputting the message; and
outputting a message requesting repair or replacement of the measuring device when the second biological data is not acquired within a preset time period.

9. The method of claim 1, further comprising:
eliminating a first valid section from the valid data range when the first biological data is not acquired during the validity time period set for a first valid section.

10. The method of claim 1, wherein the validity time period set for each of the at least one valid section is set based on the number of the biological data already included in each of the at least one valid section.

11. The method of claim 1, further comprising:
generating the valid data range based on biological data of other users, wherein the other users have characteristics corresponding to those of the user.

12. The method of claim 1, wherein the terminal is a physician terminal.

13. A apparatus comprising:
a memory configured to store at least one program; and
a processor, being controlled by the at least one program to perform:
acquiring a first biological data of a user from a measuring device;
determining whether the acquired first biological data is within a valid data range, wherein the valid data range includes at least one valid section, and a validity time period is set for each section of the at least one valid section; and
transmitting the first biological data to a terminal when the acquired first biological data is within the valid data range.

14. The apparatus of claim 13, wherein the biological data is at least one of body weight, height, blood glucose level, blood pressure, or body temperature.

15. The apparatus of claim 13, wherein the processor is further configured to perform:
updating the validity time period set for at least one valid section of the at least one valid section.

16. The apparatus of claim 13, wherein the processor is further configured to perform:
outputting a message requesting communication with the terminal when the first biological data is not included within the valid data range.

17. The apparatus of claim 13, wherein the processor is further configured to perform:
updating the valid data range based on the first biological data when information, indicating that the first biological data is valid, is received from the terminal, wherein the updating occurs despite the first biological data being not included within the valid data range.

18. The apparatus of claim 17, wherein the updating comprises adjusting the valid data range to include a new valid section including the first biological data, and setting a validity time period for the new valid section.

19. The apparatus of claim 13, wherein the processor is further configured to perform:
outputting a message requesting a re-measurement to obtain second biological data when the first biological data is not included within the valid data range.

20. The apparatus of claim 19, wherein the processor is further configured to perform:
determining whether the second biological data measured by the measuring device is acquired, after outputting the message; and
outputting a message requesting repair or replacement of the measuring device when the second biological data is not acquired within a preset time period.

21. The apparatus of claim 13, wherein the processor is further configured to perform:
eliminating a first valid section from the valid data range when the first biological data is not acquired during the validity time period set for a first valid section.

22. The apparatus of claim 13, wherein the validity time period set for each of the at least one valid section is set based on the number of the biological data already included in each of the at least one valid section.

23. The apparatus of claim 12, wherein the processor is further configured to perform:
generating the valid data range based on biological data of other users, wherein the other users have characteristics corresponding to those of the user.

24. A machine-readable non-transitory medium having stored thereon machine-executable instructions for:
acquiring a first biological data of a user from a measuring device;
determining whether the acquired first biological data is within a valid data range, wherein the valid data range includes at least one valid section, and a validity time period is set for each section of the at least one valid section; and
transmitting the first biological data to a terminal when the acquired first biological data is within the valid data range.
